(19) United States
(12) Patent Application Publication
(10) Pub. No.: US 2008/0097153 A1
(43) Pub. Date: Apr. 24, 2008

(54) METHOD AND APPARATUS FOR GRASPING AN ABDOMINAL WALL

(76) Inventors: Anthony R. Ignagni, Oberlin, OH (US); Raymond P. Onders, Shaker Heights, OH (US); James E. Gelbke, North Royalton, OH (US)

Correspondence Address: SHAY GLENN LLP, 2755 CAMPUS DRIVE, SUITE 210, SAN MATEO, CA, 94403, US

(21) Appl. No.: 11/467,025

(22) Filed: Aug. 24, 2006

(51) Int. Cl. A61B 1/01

(52) U.S. Cl. 600/114

(57) ABSTRACT

A method of delivering a tool to a peritoneal cavity including the following steps: inserting a guidewire percutaneously through an abdominal wall into a stomach; pulling the abdominal wall away from the stomach; and guiding a distal end of an endoscope with the guidewire through a wall of the stomach into the peritoneal cavity. Another aspect of the invention provides an abdominal wall grasping device having an abdominal wall attachment portion adapted to attach to an abdominal wall and a grasping element adapted to be grasped by a user to pull the abdominal wall away from a stomach.

### CROSS-REFERENCE

This application is related to the following copending patent application, filed on even date herewith: Method And Apparatus For Transgastric Neurostimulation, Attorney Docket No. 33990-702.201, which is incorporated herein by reference in its entirety.

### BACKGROUND OF THE INVENTION

Certain medical procedures performed in the peritoneal cavity require laparascopic or transgastric access to the peritoneal cavity. For example, certain neurostimulation procedures require the placement of electrodes via abdominal access. For example, US Patent Appl. Publ. No. 2005/0021102 describes a system and method for stimulating a conditioning a diaphragm through electrical stimulation of target sites on the diaphragm. The electrodes may be implanted laparascopically, using, e.g., an electrode delivery instruments such as those described in U.S. Pat. No. 5,797,923 and U.S. Pat. No. 5,472,438.

It is often desirable to electrically map possible stimulation electrode sites to find the most appropriate target site for implanting the stimulation electrode(s). An example of a mapping probe system for use with neuromuscular stimulation systems is described in US Patent Appl. Publ. No. 2005/0107860. This mapping probe is designed to be inserted laparascopically through a cannula that has been placed in the patient's peritoneal cavity through an incision in the patient's abdominal wall.

While less invasive than open surgery, laparascopic delivery of mapping and/or stimulation electrodes still requires the surgeon to make multiple incisions through the patient's skin to access to the patient's abdomen for the visualization and electrode manipulation instruments. In addition, access to certain sites within and around the abdomen may be difficult, depending on the location of the laparascopic port with respect to the target sites. While transesophageal approaches to cardiovascular and mediastinal procedures have been proposed (see, e.g., US Patent Appl. Publ. No. 2005/0148818), and while transgastric peritoneal cavity access tools have been described (see, e.g., U.S. Pat. No. 6,918,871; US Patent Appl. Publ. No. 2004/0260245; US Patent Appl. Publ. No. 2005/0277945; and US Patent Appl. Publ. No. 2001/0049497), the prior art has not adequately addressed issues relating to delivery and manipulation of transgastric tools to and in the peritoneal cavity.

### SUMMARY OF THE INVENTION

One aspect of the invention provides a method of delivering a tool to a peritoneal cavity including the following steps: inserting a guidewire percutaneously through an abdominal wall into a stomach; pulling the abdominal wall away from the stomach; and guiding a distal end of an endoscope with the guidewire through a wall of the stomach into the peritoneal cavity. In some embodiments, the method includes the step of attaching an abdominal wall grasping device to the abdominal wall prior to the pulling step. The attaching step may optionally include one or more of the following steps: inserting the grasping device into the abdominal wall; disposing the grasping device around the guidewire; sealing the grasping device around the guidewire; and/or expanding the grasping device, such as by inflating the grasping device.

Another aspect of the invention provides an abdominal wall grasping device having an abdominal wall attachment portion adapted to attach to an abdominal wall and a grasping element adapted to be grasped by a user to pull the abdominal wall away from a stomach. In some embodiments, the attachment portion includes an insertion element adapted to be inserted into an opening in the abdominal wall. The insertion portion may be expandable, such as by inflating.

In some embodiments, the device has an opening adapted to surround a guidewire. The opening may also be adapted to seal around a guidewire. In some embodiments, the grasping element has openings sized for insertion by the user's fingers.

The invention is described in more detail below with reference to the drawings.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart showing an aspect of a tissue mapping method of this invention.

FIG. 2 shows an endoscope passing into a peritoneal cavity through an opening in a stomach.

FIG. 3 shows an endoscope and mapping instrument passing into a peritoneal cavity through an opening in a stomach and retroflexed toward a diaphragm.

FIG. 4 shows an endoscope and mapping instrument passing into a peritoneal cavity through an opening in a stomach.

FIG. 5 is a flowchart showing another aspect of the transgastric mapping and electrode placement methods of this invention.

FIGS. 6A-E are schematic drawings showing a transgastric procedure according to an aspect of this invention.

### DETAILED DESCRIPTION OF THE INVENTION

One aspect of the invention provides devices and techniques for accessing the peritoneal cavity for, e.g., performing laparascopic, percutaneous and/or transgastric procedures in the peritoneal cavity. An example of such procedures is the diagnostic and therapeutic stimulation of abdominal and pelvic structures accessed through natural orifices, such as the mouth. This exemplary technique uses a standard endoscope and instruments to make a gastrostomy. The endoscope may then be passed into the peritoneal space within the peritoneal cavity and manipulated (by, e.g., bending or retroflexing) to view desired structures. A mapping instrument may be passed through one of the endoscope working channels to diagnose or identify structures. Upon completion of mapping, stimulating electrodes may be placed into target tissue using a percutaneous needle under endoscopic visualization and assistance. Alternatively, electrodes may be passed through a lumen of the endoscope. The electrode leads then may be attached to a stimulator disposed within the patient (e.g., in a subcutaneous pocket formed in the patient) and or percutaneously to an external stimulator.

The devices and techniques according to this aspect of the invention may be applied to a wide variety of disorders of the abdomen and pelvis. In many of these procedures, the target location or site is mapped prior to placement of either a trial percutaneous lead or a permanent implantable pulse generator.

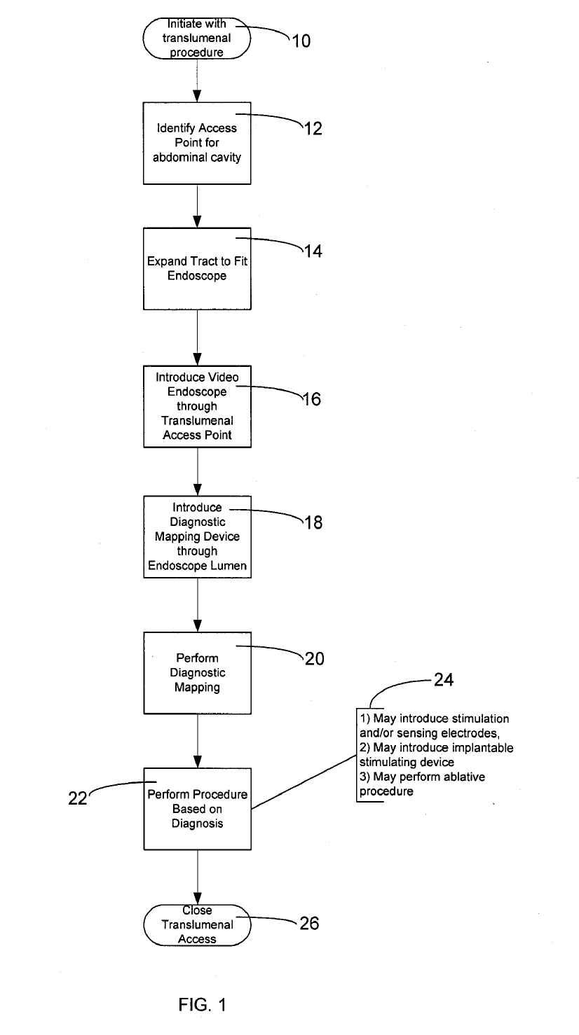

FIG. 1 is a flow chart showing an aspect of a peritoneal cavity tissue mapping method. The procedure initiates by placing an endoscope into the patient's stomach to provide translumenal access to the stomach wall, as in block 10 in FIG. 1. Using the endoscope's viewing capabilities, a peritoneal cavity access point in the stomach wall is identified (12). For example, one desirable section of stomach for this procedure may be located as far distally as is accessible by the endoscope, in a location that provides good visualization of the target abdominal or pelvic structures and that permits ready closing with a closing device.

After an opening is made in the stomach wall using a standard technique (e.g., gastrostomy), the opening is expanded to accommodate the endoscope (14), and the distal end of the endoscope is passed through the opening into the peritoneal cavity (16). After using the endoscope's viewing capabilities to locate target tissue site, a diagnostic mapping device is passed through a lumen of the endoscope so that its distal end is in the peritoneal cavity (18). Diagnostic electrical mapping may be then be performed on the target tissue (20). The mapping procedure may be used to diagnose the patient and to determine which therapeutic procedure should be performed, such as the implantation of stimulation or sensing electrodes, implantation of a stimulating device and/or tissue ablation (22, 24).

After completion of the procedure, the opening in the stomach is closed, and the endoscope is removed from the patient (26). Gastrostomy closing may be performed by placement of a percutaneous endoscopic gastrostomy (PEG) tube or by use of a ligating system, clip or T-bar device cinched to close the opening without placement of a PEG.

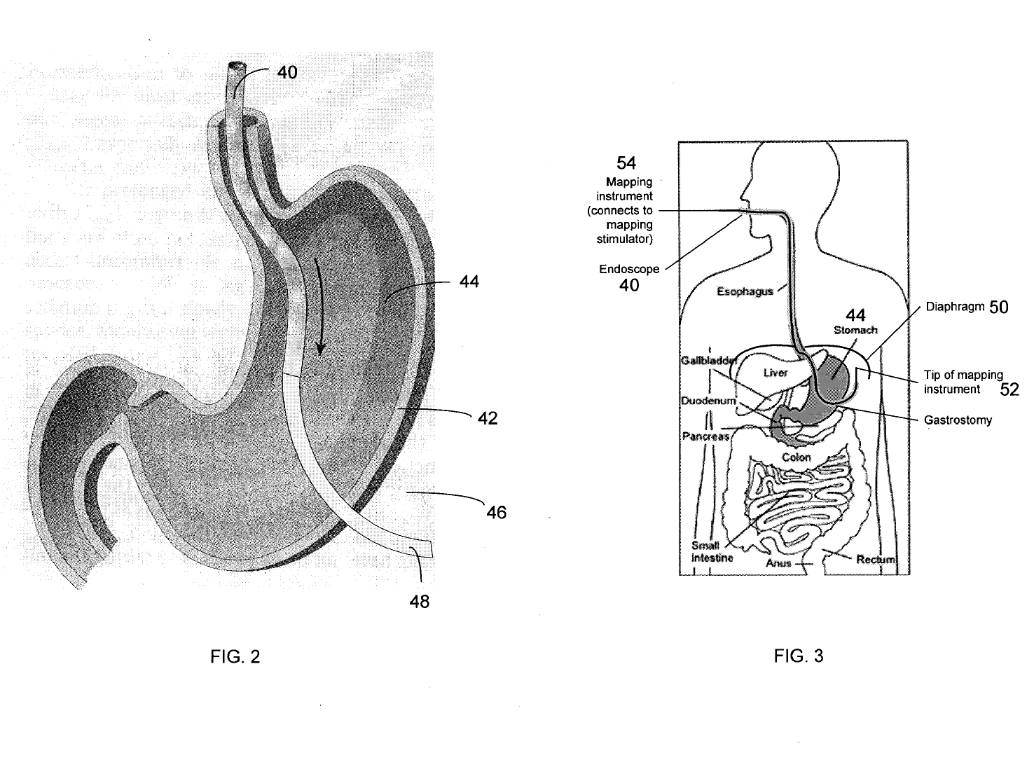

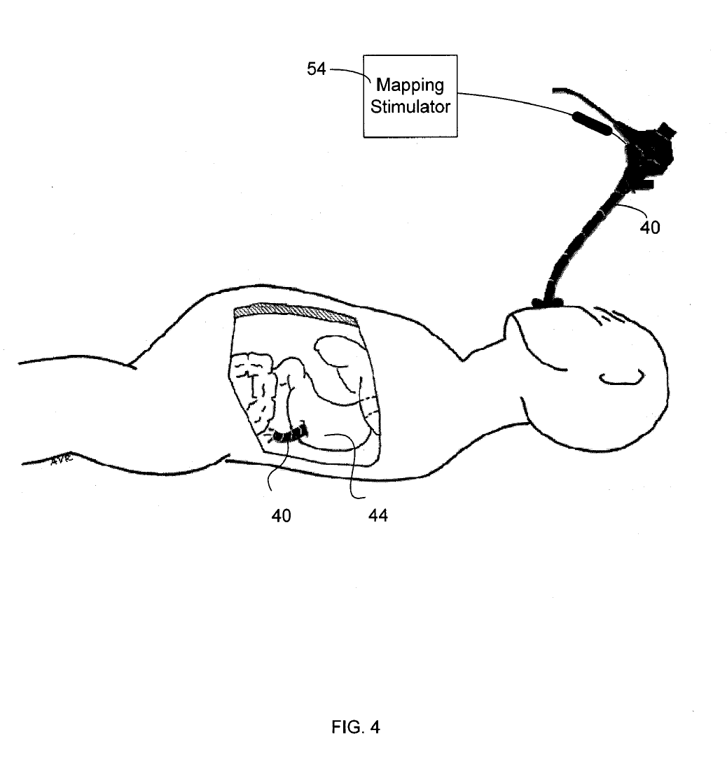

FIGS. 2-4 show an endoscope 40 passing into and through the wall 42 of a stomach 44 into the peritoneal cavity 46. The distal end 48 of the endoscope 40 may be retroflexed to view and/or provide access to, e.g., the patient's diaphragm 50, as shown in FIG. 3, which shows a mapping electrode 52 at the tip of a mapping instrument near the diaphragm. Other organs within and around the peritoneal cavity may be accessed, as shown. FIG. 4 shows how an external mapping stimulator may be connected with a mapping instrument 54. Other details regarding the formation of a gastrostomy, endoscopic access to the peritoneal cavity through a gastrostomy, and tissue mapping and stimulation in general may be found in U.S. Pat. No. 6,918,871; US Patent Appl. Publ. No. 2004/0260245; US Patent Appl. Publ. No. 2005/0277945; US Patent Appl. Publ. No. 2001/0049497; US Patent Appl. Publ. No. 2005/0021102; and US Patent Appl. Publ. No. 2005/0107860.

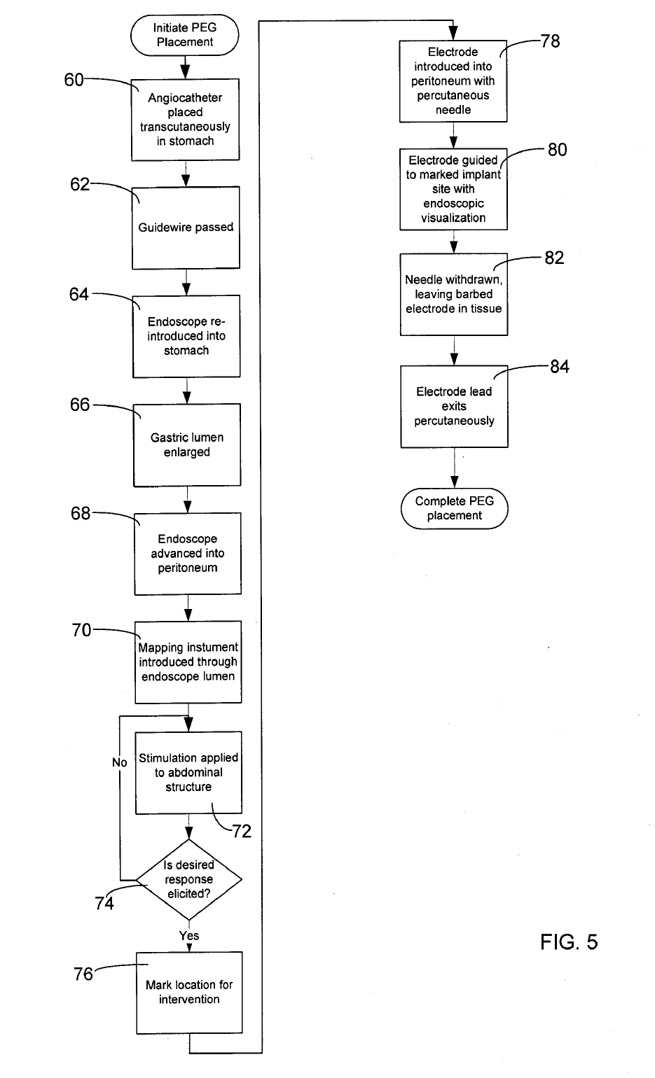

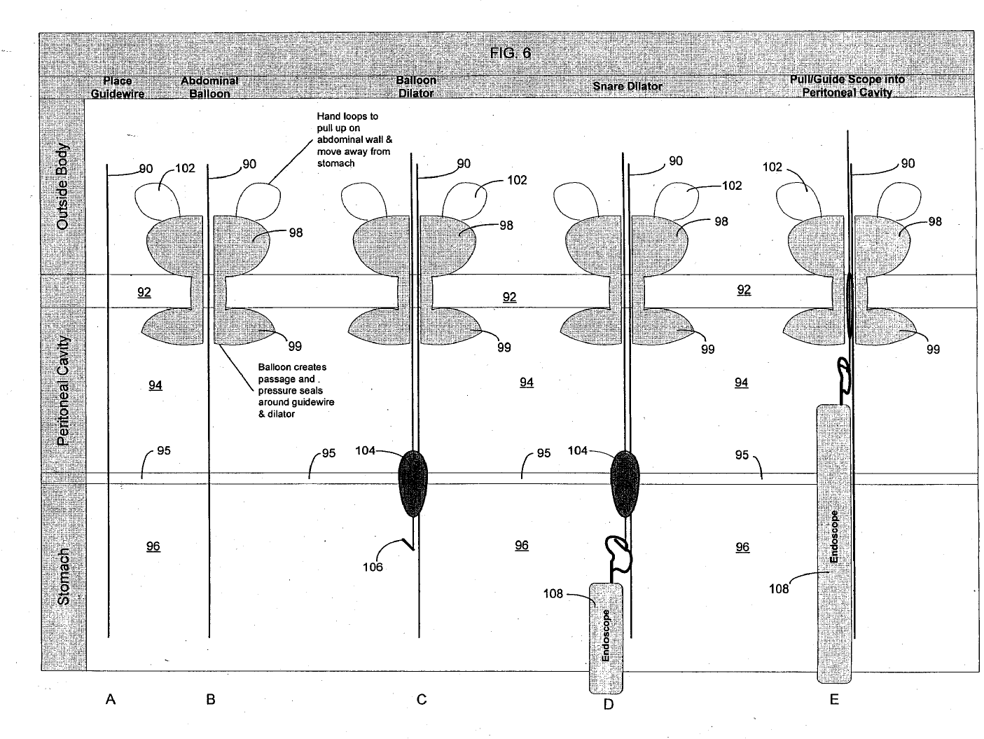

FIG. 5 is a flowchart showing an aspect of the abdominal wall grasping device and method of this invention. While the invention is described in relation to a peritoneal cavity needle placement procedure, it should be understood that the method and device of this invention can be used with other peritoneal cavity procedures. In this example, the percutaneous endoscopic gastrostomy procedure commences by placing an angiocatheter percutaneously in the patient's stomach (60). A guidewire is then passed into the stomach (62), and an endoscope is introduced (or re-introduced) into the stomach (64). The guidewire may be snared by the endoscope and pulled out of the patient's mouth, and a second guidewire may be introduced with the first guidewire to provide a guide for re-introduction of the endoscope. An overtube may also be provided with the endoscope upon re-introduction. The gastric lumen or opening formed by the angiocatheter placement is enlarged, such as with a dilating balloon passed down the guidewire (66), and the distal tip of the endoscope is advanced through the opening into the patient's peritoneum (surrounding the peritoneal cavity) (68). The second guidewire and dilating balloon may then be removed.

After movement of the endoscope (e.g., bending, retroflexing) for visualization of target structures, a mapping instrument may be passed through a lumen of the endoscope to stimulate and map target tissue within the peritoneal cavity (70, 72). Mapping stimulation responses may be monitored with instrumentation (e.g., EMG, ENG, pressure catheters, etc.) or queried from the patient (as in the case of awake endoscopy for identifying sources of chronic pain). The mapping stimulation may be a single pulse to evoke a twitch or action potential or a train of pulses to elicit a contraction or propagation of nervous system impulses. If the desired response is not elicited in the target tissue, the mapping stimulation may be repeated (74). Otherwise, if mapping is successful, the target site may be marked for electrode placement or other intervention (76).

A stimulation electrode may then introduced into the peritoneum and placed in the target tissue, such as by a percutaneous needle under visualization from the endoscope (78, 80, 82). For example, an electrode such as a barbed style electrode (e.g., a Synapse Peterson, Memberg or single helix electrode) may be loaded into a non-coring needle and penetrated through the skin. Using endoscopic visualization and (if desirable or necessary) with an endoscopic grasping tool, the electrode may be placed in the target tissue. The needle may then be removed, leaving the electrode leads extending percutaneously for connection to an external stimulation device (84). Alternatively, barbed electrodes may be placed endoscopically by introducing a small gauge needle through a lumen of the endoscope for direct placement in the target tissue. The electrode leads may be connected to a subcutaneously-placed stimulator or to a microstimulator (such as a BION® microstimulator) passed through the endoscope lumen and placed with the electrode. As yet another alternative, the electrode may be placed laparascopically using a single laparascopic port and visualization from the endoscope. This alternative may permit the manipulation and placement of larger electrodes in the peritoneal cavity.

FIGS. 6A-E show schematically some of steps of endoscopic transgastric access of the peritoneal cavity according to one aspect of the invention. In FIG. 6A, a guidewire 90 is inserted percutaneously through the patient's abdominal wall 92, through the peritoneal cavity 94 and into the patient's stomach 96. A grasping device formed as a balloon 98 with a port 100 is placed around guidewire 90 and inflated to provide a pressure seal around the guidewire, as shown in FIG. 6B. An attachment portion 99 of balloon 98 extends through the abdominal wall 92, as shown, to firmly attach the grasping device to the abdominal wall. Balloon 98 has grasping elements formed as loops 102 that may be grasped by a user's fingers to pull the abdominal wall 92 away from the stomach during the procedure. A dilator 104 is advanced in a deflated configuration through the stomach wall 95 over guidewire 90, then inflated to enlarge the stomach wall opening, as shown in FIG. 6C. A snare 106 extending from dilator 104 grasps the distal end of endoscope 108 to pull endoscope 108 into the peritoneal cavity, as shown in FIGS. 6D and 6E. Use of the grasping loops 102 to pull the abdominal wall 92 away from stomach 96 is particularly useful during this portion of the procedure. Dilator 104 may be deflated, and snare 106 unhooked from endoscope 108, to permit endoscope 108 to be used in the peritoneal cavity as described above.

The devices and methods described above may be used as part of many procedures, such as:

Diaphragm conditioning. The device and method may be used to identify phrenic nerve motor points and to implant electrodes in the diaphragm to condition or pace the diaphragm.

Gastro-esophageal reflux disease (GERD) treatment. Mapping of structures within the peritoneal cavity may be used to identify a stimulus location to elicit the best contraction of the lower esophageal sphincter (LES). A trial electrode may then be placed in the LES, and a pH sensor placed in the esophagus. After two or three days of reduced reflux, the electrode may be connected to an implantable pulse generator (IPG).

Gastroparesis. Mapping may be used to identify a stimulus location that causes stomach wall contraction. A trial electrode placed in the stomach with the electrode lead passing percutaneously outside of the patient. Upon demonstration of improved gastric emptying and reduction in nausea, the electrode may be connected to an IPG.

Morbid obesity. Mapping may be used to elicit a sensation of satiety. Trial electrodes may be placed in the stomach with the electrode leads passing percutaneously outside of the patient. The electrodes may be removed after successful weight loss or connected to an IPG if on-going treatment is required.

Chronic pain from pancreatitis treated by neuromodulation at the celiac plexus. Mapping may be used to identify a location where neuromodulation of pain takes place, and trial electrode placed into the celiac plexus. Following a demonstration of pain reduction, the temporary lead or electrode may be connected to an IPG.

Chronic pain from unspecified abdominal or pelvic pain, treated by neuromodulation at the superior hypogastric plexus, the mesenteric plexus, or other nervous system structure through which the pain pathways are mediated. Mapping may be used to identify the location where neuromodulation of pain takes place. Following demonstrated trial pain reduction, the temporary lead or electrode may be connected in to an IPG.

Nerve sparing. The devices of this invention may be used diagnostically to identify specific tissue structures a surgeon may wish to spare during a surgical procedure. A mapping stimulus may be applied to nerve or muscle tissue to elicit a response, responsive tissue noted or marked, and marked tissue may be avoided during the subsequent surgical procedure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of delivering a tool to a peritoneal cavity comprising:
inserting a guidewire percutaneously through an abdominal wall into a stomach;
pulling the abdominal wall away from the stomach; and
guiding a distal end of an endoscope with the guidewire through a wall of the stomach into the peritoneal cavity.

2. The method of claim 1 further comprising attaching an abdominal wall grasping device to the abdominal wall prior to the pulling step.

3. The method of claim 2 wherein the attaching step comprises inserting the grasping device into the abdominal wall.

4. The method of claim 2 wherein the attaching step comprises disposing the grasping device around the guidewire.

5. The method of claim 2 further comprising sealing the grasping device around the guidewire.

6. The method of claim 2 wherein the attaching step comprises expanding the grasping device.

7. The method of claim 6 wherein the expanding step comprises inflating the grasping device.

8. An abdominal wall grasping device comprising an abdominal wall attachment portion adapted to attach to an abdominal wall and a grasping element adapted to be grasped by a user to pull the abdominal wall away from a stomach.

9. The device of claim 8 wherein the attachment portion comprises an insertion element adapted to be inserted into an opening in the abdominal wall.

10. The device of claim 9 wherein the insertion portion is expandable.

11. The device of claim 10 wherein the insertion portion is inflatable.

12. The device of claim 8 further comprising an opening adapted to surround a guidewire.

13. The device of claim 12 wherein the opening is adapted to seal around a guidewire.

14. The device of claim 8 wherein the grasping element comprises openings sized for insertion by the user's fingers.
